(19) United States
(12) Patent Application Publication
(10) Pub. No.: US 2003/0171733 A1
(43) Pub. Date: Sep. 11, 2003

(54) Safety concept for a control loop for dynamically dosing medicaments

(76) Inventor: Uvo Hoelscher, Steinfurt (DE)

Correspondence Address: Elsa Keller, Siemens Corporation, Intellectual Property Department, 170 Wood Avenue South, Iselin, NJ, 08830, US

(21) Appl. No.: 10/181,749

(22) PCT Filed: Jan. 25, 2001
(86) PCT No.: PCT/DE01/00278

(30) Foreign Application Priority Data
Feb. 1, 2000 (DE) 100 04 230.9
Feb. 3, 2000 (DE) 100 04 653.3

(51) Int. Cl. A61M 31/00

(52) U.S. Cl. 604/503

(57) ABSTRACT

A control loop system for patient therapy, comprising a dosing unit which requires as an input a value which is converted into a dosing variable in order to control said therapy, whereby technical characteristics of the system or physiological characteristics of the patient or technical characteristics of the therapeutic agent such as pharmacological, physical or chemical characteristics are taken into account. The inventive control loop system also comprises a measuring device for receiving signals and measuring values of the patient or system, and further comprises a regulating device which evaluates the signals from the measuring device so that the dosing results in a desired state in said patient or in the system. The dosing device is constructed in a safe manner as an individual system. The invention is characterized in that a maximum therapy variable can be fixed and that the regulating device can modify the therapy variable in the direction of a safe state but it is limited to values which are limited by the maximum therapy variable and said safe state.

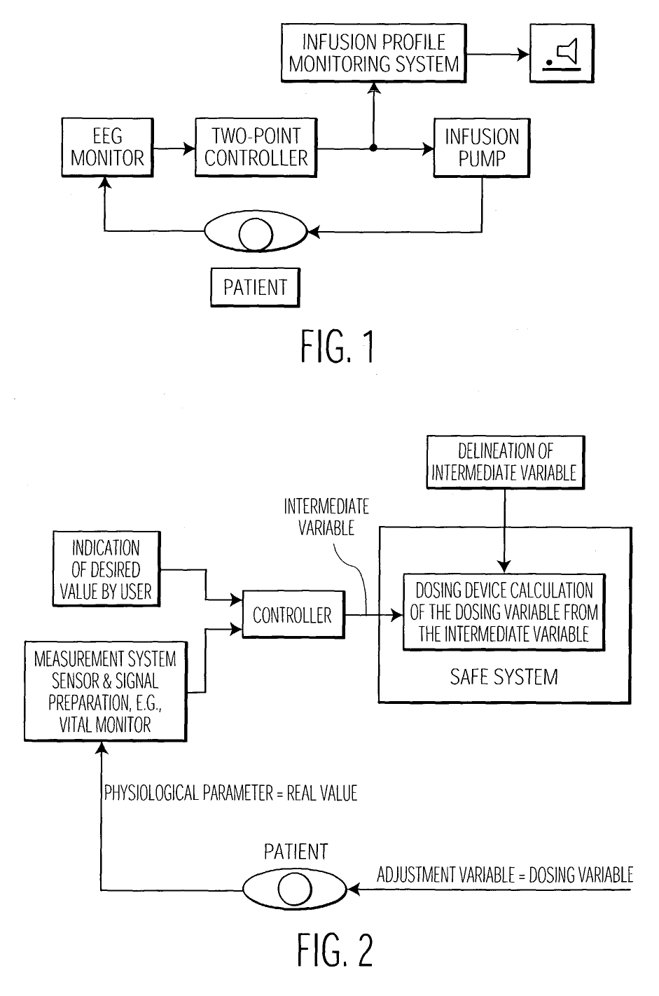

Problem Area

Systems in medical technology, which comprise several components, e.g., are not permitted to imply any hazard for the patients or the user even in the case of a first defect.

For this reason, controlled systems that are required to modify, e.g., a physiological parameter of the patient by dosing a medicament, for example, must have the following special features:

they must be assembled in a way that is safe in the event of the unnoticed occurrence of a first defect

for such a system, a safe state must be defined in the case of a failure.

The consequence of this is that, in general, the subsystems of the entire control loop either have to be assembled in the form of two channels (a second channel checks the first in terms of intactness), or checks of proper functioning have to be carried out permanently via expensive additional monitoring devices. For example, this can take place by using a monitor for the parameter that is to be stabilized, whereby the said monitor makes this parameter available to the controller in the form of a control variable/real variable, and by additionally using an independent second monitor for monitoring, whereby the said second monitor monitors the functioning of the first monitor and that of the controller. Moreover, an additional safety circuit is usually necessary that automatically initiates an alarm in the event of a discrepancy between the values from the two monitors, and transforms the system into a safe state. The entire system acquires high complexity as a result. This implies very high technical expense as well as high costs. No such system has therefore been offered commercially thus far because it cannot be produced economically using a design that is problem-free from the technical safety standpoint. At the current time, only systems with considerable safety deficiencies are coming into use, namely for research purposes (e.g., in anesthesia), whereby these systems require constant checking by an experienced physician, and they cannot therefore be registered for approval either in accordance with the regulations of the Medical Device Directive/Europe or in accordance with the regulations of the FDA/USA.

Anesthesia

Patients in the OR are brought into a hypnotic state in order to eliminate consciousness and perception during narcosis.

In narcosis, it is usual that the hypnotic part of the medicament application be controlled in accordance with the desired depth of anesthesia. The depth of hypnosis requirements, which change rapidly during narcosis, are complied with either [sic] by variable dosing by the anesthetists. The objective is to use medicaments that permit rapid adaptation of the depth of hypnosis to the required level (rapid flooding), as well as rapid awakening. These medicaments exhibit the property that dosing with them cannot usually be carried out in a constant manner as a function of time but, rather, dosing with them has to be adapted as a function of their redistribution into regions in the body that take up amounts of the medicament at different speeds and to different extents (pharmacokinetics).

Intravenous and inhalation anesthetics can be selected.

IV Anesthesia

In the case of intravenous anesthesia, the medicament is generally administered to the patient in an intravenous and continuous manner via an infusion pump. The repetitive administration of boli (larger quantities in one single step, with pauses between the doses) is not recommended because, as a result, this is accompanied by changing levels of the medicament in the blood, and changing depths of hypnosis.

Infusion pumps are not designed in such a way from the technical safety standpoint that pumping at the adjusted rate is the normal state. Infusion has to be stopped if the pump fails, or if a defect occurs. The anesthetist can continue the narcosis manually. This state is also defined as being a safe state.

BET Arrangement

In order to arrive rapidly at an adequate level of the medicament in the blood, the quantity of material to be infused was determined in accordance with the BET [bolus elimination-transfer] arrangement in the early days. An initial bolus serves for rapidly achieving an appropriate effective concentration in the blood. After this, a quantity is applied that declines as a function of time and that falls back to a value that is adequate in order to maintain narcosis. This value is achieved only after a prolonged period of time when processes involving the redistribution of the medicament in the body scarcely play a role any longer. If the narcosis has to be deepened, then a similar process has to follow on from this again. Since an exact calculation of the required quantities is not possible by means of mental arithmetic, the resulting function is a superimposition of various e-functions, dosing was usually carried out in steps in a profile that had been tried and tested in practice.

Summarizing, it can be said that a multiple of the rate, which is classified as being innocuous over a long period of time, has to be given over certain intervals of time, dosing that is desired to achieve a concentration level in the body on a short-term basis, and then maintain it. Short-term over-shooting of the safe range is permitted only in order to compensate for dynamic processes (filling the various reservoir locations in the body).

Such arrangements always find use when a medicament is to be maintained at a constant effective level. In addition to anesthesia, this is also the case with other areas of application.

TCI

Today, the availability of microprocessor controlled infusion pumps permits the ongoing calculation and adaptation of the required infusion quantity. The desired concentration level of the anesthetic agent in the body of the patient (blood level, or level in the effect compartment) is set up using such a pump. Since the characteristics of the medicaments and of the patient vary, the TCI pump usually processes the following items of patient information in order to calculate the individual infusion rate: sex, age, weight, temperature, . . . as well as the specifics of the medicaments.

Pumps that contain this function are known by the abbreviation TCI (Target Controlled Infusion). Thus, at the start of infusion and after each deepening of the level, the TCI pump automatically increases the infusion rate, for a short time, to values that are significantly above the rate that is classified as being innocuous when given over a long period of time. This is solved in such a way from the technical safety standpoint that the rate calculation that produces short-term “over control” is carried out in a two-channel manner in the pump. The TCI pump therefore represents the prior art from a technical safety standpoint.

Control Loop

The individually required concentration of the anesthetic agent in the blood varies. Clinical indications such as blood pressure, heart rate, moisture level of the skin . . . are utilized conventionally in order to ascertain the anesthetic state. More recent concepts use physiological parameters in order to determine the depth of hypnosis. The measured value can then be used in a control loop in order to achieve the necessary dosage.

For example, the electrical activity of the brain of the patient (EEG) (Schüttler, Schwilden, et al.), or the evoked potentials (Kenny) can serve as the input parameters for such control.

Ideas

The objective of this idea is to describe a concept for the design of systems from a technical safety standpoint in which the necessary two-channel nature or safety concept needs to be extended merely to a subunit of the entire system without impairing the safety of the entire system as a result.

Rate Modulation

The sedative can be administered via a conventional two-channel therapy unit, i.e., via an inherently safe unit (e.g., an infusion pump), on the basis of automated long-term sedation (e.g., monitoring the depth of sedation by a single channel monitoring device, e.g., EEG. Using the pump, the infusion rate is thereby adjusted to an innocuous value that corresponds to the usual medical dosage. With the help of the controller, the monitor can only regulate the infusion rate, e.g., via pulse breadth modulation, to values that are smaller than the rate which has been set up, e.g., by briefly stopping the pump. Since stoppage of the pump is regarded as a safe state and likewise the full infusion rate that has been set up (the medically desired rate), the entire system can only range between the two states, and is therefore safe in toto.

Modulation of an Intermediate Variable

In order to be able to control dynamic processes better, the system, as far as is possible, is required to permit dosing that is restricted as a function of time, but that is significantly above the rate that is classified as innocuous when given over a long period of time.

Use is made, for example, of a conventional TCI infusion pump, a controller, and one or more physiological monitors.

The TCI pump possesses the known adjustment possibilities with which the desired (blood) level can be set up, whereby this is termed the maximum level in the following sections. Using the TCI infusion pump, the user sets up the maximum effective level to the value that is usual in classical operation, whereby the said value represents the maximum blood level for narcosis in accordance with medical judgment.

In accordance with the invention, the TCI pump additionally possesses an input circuit that permits a decline in the blood level that has been set up. In the event of the absence of an input signal, for example, the blood level will be reduced to zero in this way. If the full input signal is applied, then only the maximum level which was set up using the TCI pump is dosed.

For example, the input circuit can function via pulse breadth modulation, or via other techniques. The only important aspect is that the effective level, which the TCI pump has as a control variable, can only vary between a safe state (e.g., zero) and the maximum level that has been set up.

In the event of a decline in the indicator of, e.g., the depth of sleep (the patient awakes), the controller evaluates the signals in such a way, using the desired control value which has been set up, that it increases the input signal for the infusion pump. If the indicator of the depth of sleep reaches the desired value again, then the controller holds the level constant once more.

Thus the controller exhibits the typical characteristic that the effective adjustment variable, in this case the blood level, can only assume values between two defined states that do not represent any hazards for the patient even on the assumption of a first defect.

This control loop advantageously reduces the supply of medicament to the required minimum in the case of a low pain stimulus or an individually high effect from small quantities of the medicament.

The safety concept is exploited in such a way in the control loop that the physician prescribes the maximum meaningful level that cannot cause damage and that guarantees the desired effect (the depth of hypnosis, for example) from normal points of view.

The controller itself can change (in this case, reduce) the level only in the direction of a safe state. Thus, in the worst case scenario, only the safe state or the maximum medically tolerable level (in this case, under-dosing or the maximum dosing, but never over-dosing) is possible in the event of a defect. Since the maximum dosage which is prescribed by the physician can be regarded as non-hazardous and zero pumping can be considered to be a safe state, the entire control loop possesses the level of safety of a two-channel concept despite the absence of redundancy in monitoring.

In addition, evaluation of the stoppage phase of the infusion pump as a function of time can reveal corresponding deviations in the expected characteristics of the infusion process, and this can indicate the presence of, or the approach of, under-dosing by initiating an appropriate alarm.

In accordance with the invention, recording of the course of dosing and the infusion profile which results therefrom represents an additional item of therapeutic information that can be made available for therapy for documentation purposes.

Thus an assembly results from this that is extremely favorable in terms of cost and makes a second physiological monitor superfluous. In addition, the controller can be constructed in a single channel manner.

1. A control loop system for patient therapy, with a dosing unit that requires, as an input, a value that is converted into the dosing variable in order to control the said therapy, and that thereby takes account of the technical characteristics of the system or the physiological characteristics of the patient or the characteristics of the therapeutic agent, such as pharmacological, physical, or chemical properties, and with a measurement device for receiving signals and measured values from the patient or from the system, and with an adjustment device that evaluates the signals from the measurement device in such a way that dosing via the dosing unit leads to the desired state in the said patient or system, whereby the dosing device is constructed in a safe manner in the form of an individual system,
characterized in that
a maximum therapy variable can be set up using the dosing device, and that the adjustment device can modify the therapy variable in the direction of the safe state though it is restricted to values that are limited by the maximum therapy variable and the said safe state.

2. System in accordance with claim 1, characterized in that the dosing unit is configured for the administration of medicaments into, for example, the blood of a person, and that the maximum dosing variable of the dosing device is adjustable, and that the adjustment device can modify the dosing variable in the direction of the safe state though it is restricted to values that are limited by the maximum dosing variable and the said safe state.

3. System in accordance with claim 1 or 2, characterized in that the dosing unit (1) is configured in the form of a TCI dosing unit (1) for the administration of medicaments into, e.g., the blood of a person, whereby the necessary dosage for achieving a constant effective level in the patient is prescribed by pharmacokinetic or pharmacodynamic calculation, and that this maximum effective level is adjustable, and that the adjustment device can modify the effective level in the direction of the safe state though it is restricted to values that are limited by the maximum dosing variable and the said safe state.

4. System in accordance with one of the preceding claims, characterized in that the adjustment device and/or the measurement device is constructed in single channel manner from the technical safety standpoint.

5. System in accordance with one of the preceding claims, characterized by a monitoring circuit that calculates approaching erroneous dosages via an evaluation with respect to time of the therapy profile, and indicates these in a timely manner by means of a display device.

6. System in accordance with one of the preceding claims, characterized by an evaluation circuit that calculates additional items of medical information regarding the therapy phases and the patient from the therapy profile that is determined with respect to time, and issues these by means of a display device.

7. System in accordance with claim 5, characterized by a monitoring circuit and display unit for the derivation of an indicator for the individual sensitivity of the patient to the medicament that is being administered.

8. System in accordance with claim 5, characterized by a monitoring circuit and display unit for the derivation from an infusion profile of items of therapeutic information regarding the waking & sleeping phases.

9. System in accordance with claim 5, characterized by a monitoring circuit and display for the derivation from the infusion profile of items of medical information regarding the therapeutic objectives.

10. System in accordance with one of the preceding claims, characterized by a test circuit for testing the integrity of the adjustment device via the dosing unit in such a way that the adjustment device has to generate a coded signal at regular intervals or, otherwise, it has to provide the pump with an alarm signal to trigger, for example, a prescribed value or to maintain the current value.

11. System in accordance with one of the preceding claims, characterized by an evaluation circuit for testing the integrity of the adjustment device via the dosing unit in such a way that the adjustment device can only shift the level between the maximum level which has been set up and a lower limiting value, whereby the output signal changes between 100% and a lower limiting value, or otherwise the dosing unit provides the user with an alarm signal.

12. System in accordance with one of the preceding claims, characterized in that the dosing unit has a memory for the characteristics of the therapeutic agent which is to be used, as well as an input arrangement for entering such characteristics into the memory, as well as an evaluation circuit that proposes or firmly prescribes limiting values for the therapy as a function of the stored characteristics.

13. System in accordance with one of the preceding claims, characterized by a test circuit for testing the integrity of the control loop via the adjustment device in such a way that the adjustment device has tables of values allocated to it with values that are typical of the sensitivity of the patient and of the system, or of the therapeutic agent in the form of the dependence of the physiological parameter on the dosing level, and that the test circuit compares the current values with these tables of values.

14. System in accordance with one of the preceding claims, characterized by a safety circuit that is allocated to the adjustment device and that allocates a sensitivity profile to the current patient or system and that constantly evaluates the subsequent course of the control process with the help of this sensitivity profile.

15. System in accordance with one of the preceding claims, characterized by a test circuit for testing the integrity of the control loop in such a way that, in the event of small and, if applicable, transitory modifications to the desired values, which are carried out in a controlled manner by the adjustment device, the test circuit compares the plausibility of the reaction of the patient, or of the system, to plausibility data.

16. System in accordance with one of the preceding claims, characterized by a test circuit for testing the integrity of the control loop in such a way that the test circuit supplies test signals, for short times in each case, to the measurement sensor of the patient monitor, and tests the measured value of the monitor in terms of its plausibility.
